(19) United States
(12) Patent Application Publication
(10) Pub. No.: US 2026/0207211 A1
(43) Pub. Date: Jul. 23, 2026

(54) SYSTEMS AND METHODS FOR FIBER OPTIC GENERATING ULTRASOUND

(71) Applicant: SonoVascular, Inc., Chapel Hills, NC (US)

(72) Inventors: Bradley James Stringer, Ridgefield, WA (US); John William Ranshaw, Charlotte, NC (US); Daniel Enrique Estay, Chapel Hill, NC (US)

(21) Appl. No.: 19/135,007

(22) PCT Filed: Nov. 30, 2023
(86) PCT No.: PCT/US2023/081916

§ 371 Date: Jun. 2, 2025

Related U.S. Application Data
(60) Provisional application No. 63/385,947, filed on Dec. 2, 2022.
(60) Provisional application No. 63/517,069, filed on Aug. 1, 2023.

(51) Int. Cl. A61B 17/22; A61B 18/22; A61B 18/24; A61B 18/26

(52) U.S. Cl. CPC A61B 17/2202; A61B 18/245; A61B 18/26; A61B 2018/2266; A61B 2018/266

(57) ABSTRACT

A device may include a catheter body. A device may include a fiber extending through the catheter body from a proximal portion of the catheter body to a distal portion of the catheter body. A device may include an ultrasound element positioned coupled to the fiber configured to receive photonic energy from the fiber, the ultrasound element having a side surface that faces a generally lateral direction with respect to a longitudinal axis of the catheter body. A device may include the side surface including at least one protrusion or recess that directs ultrasound energy laterally from the longitudinal axis of the catheter body.

### INCORPORATION BY REFERENCE TO ANY PRIORITY APPLICATIONS

This application claims the benefit of U.S. Provisional Application No. 63/517,069, filed on Aug. 1, 2023, and U.S. Provisional Application No. 63/385,947, filed on Dec. 2, 2022, which are incorporated by reference herein in their entirety. Any and all applications for which a foreign or domestic priority claim is identified in the Application Data Sheet as filed with the present application are hereby incorporated by reference under 37 CFR 1.57 for all purposes and for all that they contain.

### BACKGROUND

### Field

The present disclosure relates generally to a catheter system, and more specifically, in certain embodiments, a catheter system with an ultrasound transducer that in certain embodiments utilizes optical fibers and a light source.

### Description of the Related Art

Ultrasonic energy has been used to enhance the intravascular delivery and/or effect of various therapeutic compounds. In one system, ultrasound catheters are used to deliver ultrasonic energy and therapeutic compounds to a treatment site within a patient's vasculature. Such ultrasound catheters can comprise an elongated member configured to be advanced through a patient's vasculature and an ultrasound assembly that is positioned near a distal portion of the elongated member. The ultrasound assembly is configured to emit ultrasonic energy. Such ultrasound catheters can include a fluid delivery lumen that is used to deliver a therapeutic compound to the treatment site. In this manner, ultrasonic energy is delivered to the treatment site to enhance the penetration effect and/or delivery of the therapeutic compound.

### SUMMARY

In certain embodiments, a device configured to emit ultrasound energy comprising: a catheter body; a fiber extending through the catheter body from a proximal portion of the catheter body to a distal portion of the catheter body; and an ultrasound element positioned coupled to the fiber configured to receive photonic energy from the fiber, the ultrasound element having a side surface that faces a generally lateral direction with respect to a longitudinal axis of the catheter body; the side surface that may include at least one protrusion or recess that directs ultrasound energy laterally from the longitudinal axis of the catheter body.

In certain embodiments, the fiber connects to a light source to transmit photonic energy.

In certain embodiments, the ultrasound element is a lens.

In certain embodiments, the lens is a PDMS lens.

In certain embodiments, the lens is concave.

In certain embodiments, the lens is convex.

In certain embodiments, the lens is flat.

In certain embodiments, the lens is diffuse.

In certain embodiments, the lens forms an annular ring.

In certain embodiments, the device further including a hollow core fiber located at a center of an arrangement, the arrangement comprising the fiber.

In certain embodiments, a method to deliver ultrasonic energy to a treatment site, the method comprising: transmitting photonic energy from a light source through a fiber optic cable; generating ultrasound energy from the transmitted photonic energy at an ultrasound element connected to the fiber optic cable; penetrating a blood clot with forward firing ultrasonic energy from the ultrasound element; and radially firing the ultrasound energy from a side surface of the ultrasound element.

In certain embodiments, a device configured to emit ultrasound energy, the device comprising: a catheter body; a fiber optic cable extending through the catheter body from a proximal portion of the catheter body to a distal portion of the catheter body; and an ultrasound element coupled to the fiber optic cable configured to receive photonic energy from the fiber optic cable, wherein the ultrasound element comprises a substrate layer, a absorption layer and a resonance layer, the substrate layer comprising a structural material, the absorption layer comprising a light-absorbing material and the resonance layer comprising a thermal expansion material and wherein the structural layer is positioned closer to the fiber optic cable and the absorption layer is positioned between the structural and resonance layer.

In certain embodiments, at least a portion of the fiber optic cable is in a vacuum.

In certain embodiments, the substrate layer receives photonic energy from the fiber optic cable and causes the resonance layer to emit a plurality of ultrasonic waves.

In certain embodiments, the substrate layer is at least one of a glass material, an optical fiber core, or a PDMS material.) In certain embodiments, the resonance layer is a material with a high coefficient of thermal expansion.

In certain embodiments, the device further comprises at least one clad surrounding the fiber optic cable.

In certain embodiments, the clad has at least one divot configured to allow light to escape the fiber optic cable.

In certain embodiments, the light escapes the fiber optic cable in a forward and lateral direction.

In certain embodiments, the device further comprises a plurality of ultrasound elements positioned along the fiber optic cable.

### BRIEF DESCRIPTION OF THE DRAWINGS

FIG. 1 illustrates an example embodiment of laser generated ultrasound energy through a fiber optic cable.

FIG. 2 illustrates an example embodiment of a fiber optic generating ultrasound element.

FIG. 3 illustrates an example embodiment of a fiber optic generating ultrasound element inside a blood clot.

FIG. 4 illustrates an example embodiment of a fiber optic cable.

FIG. 5 illustrates an example embodiment of a fiber optic cable.

FIG. 6 illustrates an internal view of an example embodiment of a fiber optic cable and a generating ultrasound element with a guidewire.

FIG. 7A illustrates an example embodiment of a fiber optic cable with various cladding features.

FIG. 7B illustrates an example embodiment of a fiber optic cable with various tip types.

FIG. 8A illustrates an example embodiment of a light source emitting light transmitted through fiber optic cable that produced laser generated ultrasound energy.

FIG. 8B illustrates an example modified embodiment of an ultrasound element.

FIG. 9A illustrates an example embodiment of an external view of a fiber optic cable surrounded by an ultrasound element.

FIG. 9B illustrates an example embodiment of an internal view of a fiber optic cable surrounded by an ultrasound element.

FIG. 10A illustrates an example embodiment of a side view of a plurality of fiber optic cables each surrounded by an ultrasound element.

FIG. 10B illustrates an example embodiment of an isometric view of a plurality of fiber optic cables each surrounded by an ultrasound element.

FIG. 10C illustrates an example embodiment of a front view of a plurality of fiber optic cables each surrounded by an ultrasound element.

FIG. 11A illustrates an example embodiment of an internal view of fiber optic cable surrounded by an ultrasound element.

FIG. 11B illustrates an example embodiment of an external view of fiber optic cable surrounded by an ultrasound element.

FIG. 11C illustrates an example embodiment of an internal view of fiber optic cable surrounded by an ultrasound element.

FIG. 11D illustrates an example embodiment of an external view of fiber optic cable surrounded by an ultrasound element.

FIG. 12 illustrates an example embodiment of a fiber optic cable surrounded by a plurality of ultrasound elements.

FIG. 13 illustrates an example embodiment of a fiber optic cable with tilted fiber Bragg gratings.

FIG. 14 illustrates an example embodiment of a fiber optic cable with uniform fiber Bragg gratings.

### DETAILED DESCRIPTION

As used herein, the term “ultrasonic energy” or “ultrasound energy” is used broadly, includes its ordinary meaning, and further includes mechanical energy transferred through compression and rarefaction waves with a frequency greater than about 20 kHz. Ultrasonic energy waves can have a center frequency between about 20 kHz and about 22 MHz. In some embodiments, ultrasound transducers may use a multiplicity of ultrasound energy frequencies to enhance cavitation. For example, multiple ultrasound transducers can be used in parallel or series to enhance cavitation. Additionally, the ultrasound transducers may operate at different frequencies to produce a broadband of frequencies. As used herein, the term “catheter” is used broadly, includes its ordinary meaning, and further includes an elongated flexible tube configured to be inserted into the body of a patient, such as into a body part, cavity, duct, or vessel (both arterial vessels and venous vessels). As used herein, the term “therapeutic compound” is used broadly, includes its ordinary meaning, and encompasses drugs, medicaments, dissolution compounds, genetic materials, and other substances capable of effecting physiological functions. A mixture comprising such substances is encompassed within this definition of “therapeutic compound”. As used herein, the term “fiber optic cable” is used broadly, includes its ordinary meaning, and further includes an elongated flexible cable configured to transmit photonic energy and be inserted into the body of a patient, such as into a body part, cavity, duct, or vessel (both arterial vessels and venous vessels).

As expounded herein, ultrasonic energy is often used to enhance the delivery and/or effect of a therapeutic compound. For example, in the context of treating vascular occlusions, ultrasonic energy has been shown to increase enzyme-mediated thrombolysis by enhancing the delivery of thrombolytic agents into a thrombus, where such agents lyse the thrombus by degrading the platelets of the thrombus and the fibrin matrix. The thrombolytic activity of the agent is enhanced in the presence of ultrasonic energy in the thrombus because, for example, the ultrasonic energy can expose additional binding sites for the therapeutic compound. However, it should be appreciated that the present disclosure should not be limited to the mechanism by which the ultrasound enhances treatment unless otherwise stated. In other applications, ultrasonic energy has also been shown to enhance transfection of gene-based drugs into cells, and augment transfer of chemotherapeutic drugs into tumor cells. Ultrasonic energy delivered from within a patient's body has been found to be capable of producing non-thermal effects that increase biological tissue permeability to therapeutic compounds by up to or greater than an order of magnitude.

Ultrasound energy can be generated by various mechanisms of inducement. For example, ultrasound energy can be optoacoustically generated which can also be referred to as photoacoustically generated. In some examples, the inducement or generation may be used in combination with a fiber optic cable that may transmit the photonic energy to an ultrasound element; this may lead to the generation of ultrasonic energy radiated from the ultrasound element. In some examples, a light source, for example, a laser, high power LED, or the like, may generate light which may be transmitted through the fiber optic cable to the ultrasound element. The ultrasound element may be a lens or other material that may produce ultrasonic energy. In some examples, photonic energy transmits through a fiber optic cable and may contact a lens; this contact can result in a generation of ultrasound energy. The photonic energy may be absorbed by the lens and cause generation of ultrasonic energy. In some examples, the lens may be an ultrasound producing lens. For example, the lens may be a polydimethylsiloxane (“PDMS”) lens. Excitation of the lens may cause the lens to rapidly undergo thermo-elastic expansion then return to a quiescent state. Upon the return to the quiescent state, the ultrasound lens may produce vibrations that may be within an ultrasonic frequency range. In some examples, the PDMS lens may resonate at a higher frequency than other ultrasound transducers (e.g., piezoceramic transducers). In some examples, the PDMS lens can resonate at a frequency within a range of 20 kHz-22 MHz. In some examples, the wavelength of the higher frequency ultrasound may be smaller than lower frequency ultrasound. In some examples, the wavelength of the higher frequency ultrasound may be 0.076 mm to 0.76 mm. In a preferred embodiment, the wavelength of the higher frequency ultrasound may be approximately 0.158 mm. This may enhance the interaction between ultrasonic energy and the therapeutic compound, for example, ultrasonic contrast-enhancing microbubbles and/or metastable phase change nanodroplets. Additionally, this may be an efficacious manner to cause the microbubbles and/or metastable phase change nanodroplets to undergo inertial and/or stable cavitation.

The PDMS lens may generate ultrasound energy upon excitation, or absorbing light or photonic energy. Specifically, the fiber optic cable can terminate at a lens which is rapidly heated and expanded so as to vibrate. In this manner, high-frequency ultrasound can be generated using pulsed laser irradiation on light-absorbing materials (i.e., the lens or transducer) which in turn causes thermo-clastic volume expansion at a high frequency. In some examples, the PDMS lens can produce an ultrasound radiation field that is of higher power or more powerful than other ultrasound transducers (e.g., piezo transducers). This may more effectively drive the microbubbles and/or metastable phase change nanodroplets into stable and/or inertial cavitation. While the current disclosure is generally directed to optoacoustically or photoacoustically generated ultrasound, certain features of this disclosure may find application with respect to embodiments that use an ultrasound element that may be a piezoelectric material that may convert electrical charges and generate ultrasound energy. The generated ultrasonic energy may be used to treat a treatment site of a patient. For example, the ultrasonic energy may be used to treat a blood clot or pulmonary embolism at a treatment site of a patient. While ultrasonic energy may be used in context of treating a thrombus at the treatment site of the patient, the ultrasonic energy may have any therapeutic effect. In some examples, the ultrasonic energy may be used to treat in stent restenosis.

In some examples, the emitted ultrasonic energy can be focused or unfocused. Focused ultrasound energy can be used to treat a targeted area at the treatment site, for example a blood clot that may be located at the treatment site. Additionally, focused ultrasonic energy may minimize the risk of damage to surrounding tissue at the treatment site. Unfocused ultrasound energy can be used to treat an area by penetrating a target that may be located at the treatment site. For example, the target may be a blood clot. In some examples, the unfocused ultrasound energy can be forward facing. By penetrating the target, the fiber optic cable and/or ultrasound element may be able to enter the blood clot. This can enhance the effectiveness of the ultrasound treatment delivered to the blood clot, for example, by breaking down the target and delivering ultrasonic energy to an internal side of the target. In some examples, the focused ultrasonic energy can be directed radially outward from the lens. The focused ultrasound energy may be delivered to the target from inside the target. In other words, the focused ultrasound energy may treat the target, or for example, a blood clot, from the inside of the target outward. This can deliver the ultrasonic energy in a radial manner. For example, the ultrasonic energy can target the blood clot from the center of the blood to outward in approximately a 0 degree to approximately 360 degree range. This may allow for the ultrasound energy and/or therapeutic agent to treat the target, or blood clot, more effectively.

In some examples, the ultrasound element can include additional radiation elements in the form of protrusions, divots, apertures, windows, etc. that can allow for the ultrasonic energy to radiate radially outward. The radiation elements can relate to changes in frequency, for example allowing dual/multi frequency capabilities. The divots may be located at various locations of or on the ultrasound element. In some examples, the divots may be arranged in a symmetric or asymmetric pattern on the ultrasound element. In some examples, the divots of the ultrasound element may enter the inside of the target. The divots may allow focused ultrasonic energy to be delivered to the target. In some examples, the ultrasonic energy can be delivered to the target from the divots of the ultrasound element in a radially outward manner. The outer thickness of the ultrasound element can be variable to allow for multiple frequencies. The layers of the ultrasound element can alternate in thickness. The outer thickness and layer thickness may be an important design criteria due to the relationship of the frequency to thickness.

In some examples, the device can include additional mechanical features to improve mechanical breakdown of the target. For example, the ultrasound element can include spikes, blades, actuators, needles, or a screw tip. The mechanical breakdown features can be positioned at a distal end of the device. Advantageously, these features can assist in breaking apart a blood clot.

In some examples, the focused ultrasound energy may fire simultaneously, before or after the firing of the unfocused ultrasound energy. The mode and/or time of firing of the ultrasound element may be predetermined. In other words, the timing of firing the focused ultrasound energy and the timing of firing the unfocused ultrasound energy may be predetermined. The focused ultrasound energy may be used to treat the blood clot from inside the blood clot and radially outward. This may treat the blood clot from the inside out, which may maximize the effectiveness of the treatment. Additionally, this may minimize the damage and the risk of any damage to surrounding tissue at the treatment site.

In some examples, the fiber optic generating ultrasonic element can be used in combination with a catheter or catheter system. For example, the fiber optic cable may be integrated with a guidewire of a catheter or catheter system. The guidewire can control the movement and/or deflection of the fiber optic cable. The guidewire can assist a user in delivering the treatment to the treatment site of a patient. Similarly, the guidewire can help to control where the ultrasound energy treatment may be delivered. In some examples, the fiber optic cable may include a hollow core fiber. The hollow core fiber may provide a space the guidewire to integrate with the fiber optic cables.

Use of an ultrasound catheter to deliver ultrasonic energy directly to the treatment site mediates or overcomes many of the disadvantages associated with ultrasonic treatment such as low efficiency, surrounding tissue damage, and significant side effects caused by high therapeutic agent dosage levels. Additionally, use of an ultrasound element operably connected to a fiber optic cable to directly deliver ultrasonic energy in a radially outward manner may overcome disadvantages associated with ultrasonic treatment, such as low efficiency, surrounding tissue damage, and significant side effects caused by high therapeutic agent dosage levels. Furthermore, in certain examples, the ultrasound element that may be operably connected to a fiber optic cable can improve the reduction of thrombus burden and minimize bleeding complications, blood loss, and vessel wall damage due to multiple passes resulting from alternative thrombectomy devices.

In some examples, at least a portion of the fiber optic cable may be coupled to, encircled by, surrounded by, or encased by the ultrasound element, for example, via direct adhesion, optical adhesive, or the like. For example, the fiber optic cable may emit photonic energy to be received by the ultrasound element. In some examples, at least the portion of the fiber optic cable that is coupled to encircled by, surrounded by, or encased by the ultrasound element may be in a vacuum environment. In some examples, the vacuum environment may be provided by the ultrasound element coupling to the fiber optic cable. In some examples, the ultrasound element may be circular, elongated, elliptical, rectangular, ovular, or the like. The ultrasound element may be divided into various layers. The various layers may enhance the generation and radiation of ultrasonic waves. The ultrasound element may include layers such as a substrate layer, an absorption layer, and a resonance layer. The substrate layer(s) may include structural materials that provide structure to the ultrasound element and can include but are not limited to materials such as glass, PDMS material, or itself be an optical fiber core that as explained below can be used to increase the surface area at the end of the fiber optic cable. In some examples, substrate layer may be made of a material that has sufficient shaping capabilities and allow for high transmission of light (i.e., may allow for a transmission of light greater than approximately 75%). In some examples, structural materials may provide structure to the ultrasound element by allowing the resonance layer to vibrate bidirectionally (i.e., vibrating in a forward and backward direction). This may increase the efficiency of the ultrasound element. Two thickness layers may output a combined waveform of dual/multi-frequency. Additionally, in some examples, the structural materials may provide structure to the ultrasound element by providing greater surface area to the substrate layer which can enact power more diffusely for the ultrasound element. In other words, the structural materials provide additional layers to the substrate layer that may provide bulk to the substrate layer. This may increase the overall efficiency of the ultrasound element. Additionally, the bulkiness of the substrate layer may help to avoid atraumatic issue with use of the catheter. Furthermore, the bulkiness of the substrate layer may also provide beam focusing capabilities as needed and dual/multi-frequency benefits. The absorption layer may be made of light absorbing materials that have high light absorption coefficients. Example light absorbing materials include but are not limited to a carbon-based dopant with a high absorption coefficient (e.g., carbon nanotubes (single-walled or multi-walled), candle soot nanoparticles, carbon nanofibers, or carbon black). In some examples, the high absorption coefficient may be within a range of approximately 1 1/μ to approximately 32 1/μ. The absorption layer may have a thickness of approximately less than 2 mm, but not so thin that containment of all or substantially all light is not achieved. For example, at least 95% of light may be contained. In other words, the absorption layer may have a thickness that is thick enough or large enough to contain any and/or all light or substantially all light. The resonance layer can include a thermal expansion material having a high coefficient of thermal expansion and may include, but is not limited to, be a PDMS material, EEA material, EVA material, FEP material, PB material, CA material, PVDF material, PA material, PE material, Paraffin material, or PP material. The coefficient of thermal expansion may between a range of approximately 100-400×10⁻⁶ 1/k. The thickness of resonance layer may be an important design criteria due to the relationship of the frequency of resonance to thickness. For example, the thickness of the resonance layer in certain embodiments is advantageously not greater than or exceed 2 mm, which may affect the dependency of the frequency of resonance to thickness. Absorption and resonance layers may be combined with a total thickness that will not exceed the combination of the prior stated values, which in certain embodiments is 4 mm.

In some examples, the fiber optic cable may include cladding. For example, the cladding may surround the core of the fiber optic cable, the cladding may be flush with the fiber optic cable, and/or the cladding may be coupled to the fiber optic cable. In some examples, the cladding may extend the length of the fiber optic cable. In some examples, the cladding may be intermittently spaced along the length of the fiber optic cable. In some examples, the cladding may be coupled to the fiber optic cable at the distal end of the fiber optic cable. The cladding may exist in between the core of the fiber optic cable and the ultrasound element. In other words, the ultrasound element may surround or encircle the cladding in addition to the fiber optic cable. In some examples, the ultrasound element may surround each clad individually. This may increase the flexibility of the fiber optic ultrasound generating device. Similarly, this may allow the fiber optic ultrasound generating device to be conformable to patient-specific anatomy. In this manner, the individual ultrasound element may be individually controlled. For example, some, but not all, ultrasound elements may receive photonic energy at a certain time. Similarly, some but not all, ultrasound elements may emit acoustic energy at a certain time. This may be controlled by which of ultrasound elements receive photonic energy at a given time. This may increase the effectiveness of the ultrasonic treatment.

In some examples, the cladding may include at least one divot, scratch, etch, indent, gratings, or the like. For example, the divot may allow the photonic energy to be released from the fiber optic cable at various points along the fiber optic cable. The shape and/or arrangement of the divot may allow for the photonic energy to be fired in any direction. For example, the shape and/or arrangement of the divot may allow for the photonic energy to be fired in an approximately 360 degree direction. In some examples, the divots may be arranged in a predetermined pattern or a random arrangement. In some examples, the divots may extend any length of the cladding or be concentrated in at least a portion of the cladding. The divots may be included in the cladding that is surrounded by the ultrasound element. In this manner, the ultrasonic energy is emitted in various directions. For example, the ultrasound element may be laterally or forwardly firing. Similarly, the ultrasonic energy is delivered to the patient at various and/or multiple locations. This can increase the effectiveness and/or efficiency of the ultrasonic treatment.

In some examples, the divots included in the cladding and/or the cladding, alone or in combination may control the emission of the photonic energy. In this manner, a constant and consistent spatiotemporal heating of the ultrasound element. This can increase the efficiency of the device.

In some examples, more than one fiber optic cable may be included within the device. In this manner, each fiber optic cable may be grouped and included within a larger fiber optic cable. This may allow for each of the fiber optic cables to be controlled individually. Similar to the discussion above, some, but not all, fiber optic cables may emit photonic energy to be received by their respective ultrasound elements at a certain time. Similarly, some but not all, fiber optic cables may emit photonic energy to be received by their respective ultrasound element at a certain time. This may be controlled by which of ultrasound elements of each individual fiber optic cable receive photonic energy at a given time. This may allow the treatment to be more targeted. This may also increase the effectiveness of the ultrasound treatment.

In some examples, the device may include more than one fiber optic cable each surrounded by an ultrasound element, respectively. The fiber optic cables in combination with the ultrasound elements may be arranged in a co-planar or non-co-planar arrangement. For example, the fiber optic cables in combination with the ultrasound elements may be arranged in a non-co-planar arrangement to adjust or control the phase relations between the ultrasound elements. This may improve the effectiveness of the treatment by, for example, better targeting the treatment site and/or improving the destruction of the thrombus at the treatment site. The phase relations between the ultrasound elements may be adjusted based on an ultrasound wavelength (λ/n), where n may equal the number of ultrasound elements in the transducer. For example, n may be equal to 1, 2, 3, 4, 5, 6, 7, 8, etc.

In some examples, the device may include more than one light source, for example, more than one laser. In some examples, the device may include a second laser. In some examples, a second fiber optic cable may receive and transmit the generated laser from the second laser source. The second fiber optic cable may be customizable and designed as a single-mode or multi-mode fiber. The second fiber optic cable may have a diameter of approximately 250 μm. The second laser may be a low power laser of, for example, approximately 200 nW. The second laser may be a solid-state laser diode. There are many advantages to use of the second laser. For example, the second laser may provide a feedback control while remaining inexpensive, small, lightweight, have passive convective cooling, use modest power wattage, etc. The second laser may have a wavelength of approximately 850 nm-975 nm.

In some examples, the second laser may provide a feedback control for the other lasers used within the device. For example, the second laser may monitor a temperature of the blood and/or tissue surrounding the insonation zone. Monitoring the temperature of the insonation zone may help to avoid overheating of the tissue, blood, and/or laser. Additionally, this may allow the device to be driven closer to the target site and therefore providing a more directed treatment to the target site. This may reduce the overall time of the procedure, reduce the amount of drugs delivering to the target site, reduce the risk of damage caused to surrounding tissues, increase the efficiency of the treatment, etc. In some examples, single point or multi point fiber Bragg grating sensors may be used. The second laser may also monitor pH, flow, pressure, force, torsional measurements, a distance between the device and/or catheter and the target site, color reflectance spectroscopy, flex of the fiber optical fiber, chemical fluorescence, etc.

The second laser may monitor the pressure or force that the fiber optic cable is applying to the target site. This may help to ensure the fiber optic cable is not damaging surrounding tissue. This may also help to ensure that the fiber optic cable is efficiently treating the target site. The second laser may also provide feedback regarding the color reflectance spectroscopy of the surrounding blood or tissue. For example, the second laser may provide feedback regarding the blood color (SᵥO₂), the condition of the clot including but not limited to the age, degradation, thickness, strength, toughness, etc. of the target tissue. The second laser may also assist in guiding the device to the target site and provide feedback regarding the tortuosity of the vessel that the device may travel through. Additionally, the second laser may also detect gas released during inertial cavitation of the microbubbles, chemicals related to lysis, etc.

In some examples, the fiber optic generating ultrasonic element may be used in combination with a catheter or catheter system. The catheter system may include a lumen that allows for delivery of a therapeutic compound to the target area. This may more efficiently and effectively treat the blood clot. In some examples, ultrasonic contrast enhancing microbubbles and/or metastable phase change nanodroplets may be added to the therapeutic compound. For example, the microbubbles and/or metastable phase change nanodroplets may be driven to stable and/or inertial cavitation by the ultrasound energy delivered by the ultrasound catheter. The force of radiation may be the application of an acoustic force that pushes or forces the microbubbles and/or metastable phase change nanodroplets away from the transducer or away from the distal end of the transducer. This, in combination or not with cavitation, may assist the microbubbles and/or metastable phase change nanodroplets in entering and traveling through the channels created as a result of the force. Any number of the microbubbles and/or metastable phase change nanodroplets may or may not respond to the delivered ultrasound energy, however, all bubbles will be affected by radiation force, driving them distally away from the transducer face. This may result in any number of the microbubbles and/or metastable phase change nanodroplets stably vibrating throughout the treatment. For example, any number of the microbubbles and/or metastable phase change nanodroplets may be stable for a period of time and then undergo inertial cavitation. As another example, any number of the microbubbles and/or metastable phase change nanodroplets may undergo inertial cavitation once the ultrasound energy is delivered. The microbubbles and/or metastable phase change nanodroplets may be added together or individually. Some or all of the microbubbles and/or metastable phase change nanodroplets may be affected by the oscillation of some of all of the surrounding microbubbles and/or metastable phase change nanodroplets. The microbubbles and/or metastable phase change nanodroplets may be used in combination with or carry therapeutic agents, such as blood clot lysing agents. The microbubbles and/or nanodroplets can enhance the effectiveness of the ultrasound energy delivered to the treatment site. For example, ultrasound energy can be applied to the blood clot, including the microbubbles and/or metastable phase change nanodroplets within and/or surround the clot, causing the metastable phase change nanodroplets or microbubbles to oscillate, cavitate (both inertially and non-inertially), vaporize, and lyse the clot from within and/or surrounding the clot. Bioeffects may be achieved in result of the activation of the microbubbles from the ultrasound, which can include sonoporation, microstreaming and/or microjetting. The use of therapeutic agents (e.g., rt-PA) in combination with microbubbles and/or metastable phase change nanodroplets to enhance sonothrombolysis can allow blood clots to be lysed more effectively and allow for the use of a reduced dosage of the therapeutic agent while the effectiveness of the treatment remains enhanced. In some examples, vasodilation of blood vessels may occur as well. This can help or promote autolyzing, which may increase the effectiveness of the treatment. The methods and systems for microbubbles and/or metastable phase change nanodroplets are further described in US 2020/0405258 and US 2021/0007759 incorporated herein by reference.

The techniques disclosed herein can find utility with a wide variety of ultrasound catheters in addition to the ultrasound catheter embodiments described here. Certain of the techniques disclosed herein are compatible with ultrasound catheters and/or ultrasonic elements.

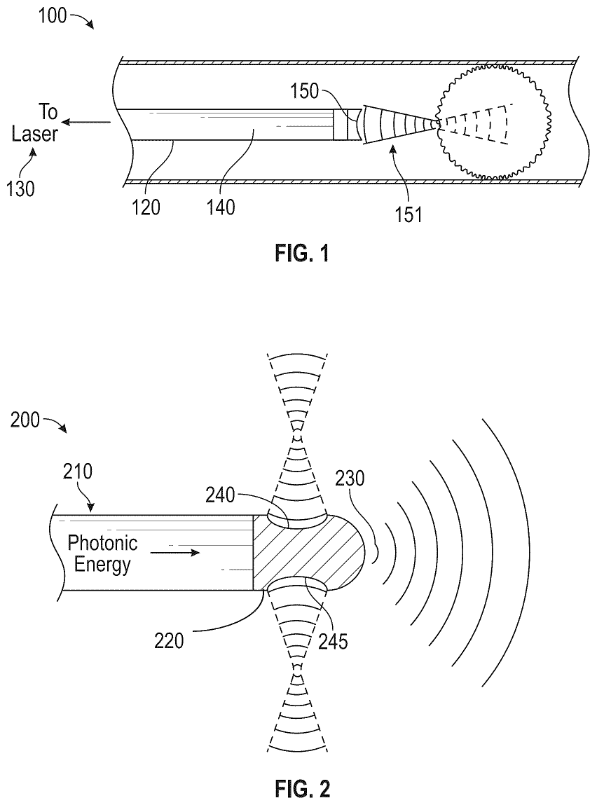

With reference to the illustrated embodiments, FIG. 1 illustrates an example of a device 100 including an ultrasound element 150 that may be operably connected to a fiber optic cable 120. The device 100 may be used in a vessel, for example the Pulmonary Artery. The fiber optic cable 120 may be of various lengths to sufficiently deliver ultrasound energy to a treatment site or target. The fiber optic cable 120 may be composed of optical fibers. The fiber optic cable 120 may be connected to a laser source 130. The laser source 130 may emit photonic energy 140, for example green laser light, that is transmitted through the fiber optic cable 120. In some examples, the laser source 130 may produce photonic energy 140 at various wavelengths. For example, the laser source 130 may produce green laser light having a wavelength of 532 nm. As shown in FIG. 1, the fiber optic cable 120 may transmit the laser light to an ultrasound element 150 that may produce ultrasound energy upon absorption of the laser light which can cause the ultrasound element to rapidly expand and then contract. The ultrasound element 150 may be located at an end point of the fiber optic cable 120. In some examples, the ultrasound element 150 may be located at any point along the length of the fiber optic cable. The ultrasound element 150 may form a lens. The lens 151 may be an ultrasonic energy converging lens, for example, a PDMS lens. The lens 151 may be concave, convex, flat, diffuse, etc. The lens 151 can be a plano-convex PDMS lens. The lens 151 can be a powered lens with variable thickness, for example with a varying energy beam pattern. The shape of the lens 151 may affect the generation or direction of deliverance of the generated ultrasound energy. In addition to the lens 151, the ultrasound element 150 may include at least one additional radiation element in the form of a protrusion, divot, aperture, window, etc. (not illustrated in FIG. 1). The radiation element may be located at various locations on the ultrasound element 150. This may allow for the ultrasound element to transmit ultrasound energy radially. The ultrasound energy may be delivered to or emitted towards a target or (i.e., a blood clot) within a patient. For example, the blood clot may be located within an artery, vein, vessel, etc.

With reference to FIG. 2, another example embodiment of a device 200 including ultrasound element operably connected to a fiber optic cable is shown. As shown, photonic energy may be transmitted through a fiber optic cable 210. The fiber optic cable 210 may connect to an ultrasound element 220. The ultrasound element 220 may be located at an end of the fiber optic cable 210. The ultrasound element 220 may be a lens, for example a PDMS lens. The ultrasound element 220 can be concave, convex, flat, diffuse, etc. Additionally, the ultrasound element 220 may be of various shapes and sizes. As shown in FIG. 2, the ultrasound element 220 includes a front face 230 and a first side face 240 and a second side face 245. The front face 230 is convex or rounded, which can provide forward facing ultrasound energy. The forward facing ultrasound energy may be unfocused ultrasound energy. The first side face 240 and the second side face 245 include an annular ring that generates side firing ultrasound energy. The side firing energy may be focused ultrasound energy. The focused and unfocused ultrasound energy may be fired at various wavelengths 2.

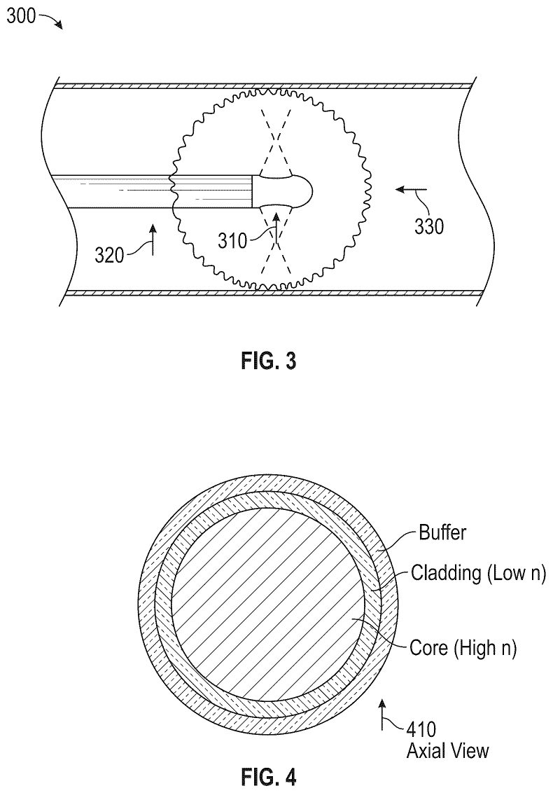

FIG. 3 illustrates the device of FIG. 2 positioned inside a blood clot. The device 200 can be used in a vessel, for example in the Pulmonary Artery. The device 200 may include an ultrasound element 220 operably connected to a fiber optic cable 210. As shown, various lengths and/or portions of the ultrasound element 220 can be entered inside of a blood clot 330. Additionally, various lengths and/or portions of the fiber optic cable 210 may be entered inside of the blood clot 330. For example, 0-100% of the ultrasound element 310 may enter the blood clot 330. Similarly, 0-100% of the length of the fiber optic cable 210 may enter inside of the blood clot 330. The percentage of the ultrasound element 310 that may enter the blood clot 330 may correspond with the amount of ultrasound energy that is delivered to the blood clot 330. In other words, if the maximum percentage of the ultrasound element 220 is entered into the blood clot 330, then the maximum amount of ultrasound energy may be delivered to the blood clot 330. Similarly, a portion of the fiber optic cable 210 may enter into the blood clot 330. This may ensure the maximum amount of ultrasound energy is delivered to the treatment site or blood clot 330. Additionally, this may minimize the damage or risk of damage of surrounding tissue at the treatment site.

FIG. 4 illustrates an axial view of an example embodiment of a fiber optic cable. As shown, the fiber optic cable 410 may have various layers. The fiber optic cable 410 may have a core. Light can be emitted through the core. In some examples, the core may be glass. The core and the cladding may have different indices of refraction, n. The fiber optic cable 410 may include cladding. In some examples, the cladding may be glass. The fiber optic cable 410 may further include a buffer. In some examples, the buffer may be a polymer. This embodiment of a fiber optic cable 410 can be used in any of the embodiments described herein.

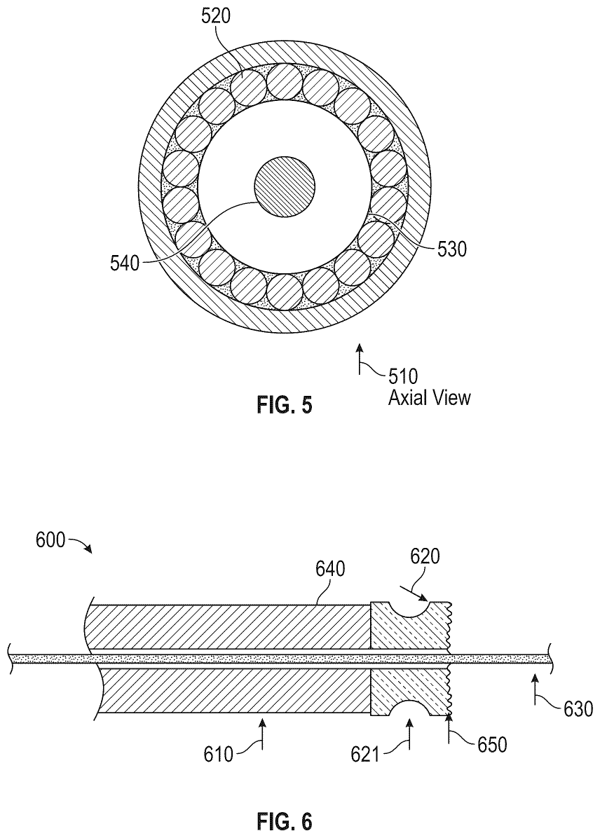

FIG. 5 illustrates an axial view of an example embodiment of a fiber optic cable 510. As shown, the fiber optic cable 510 may include various layers. The fiber optic cable 510 may be a hollow core fiber. The hollow core fiber 510 may include an internal arrangement of at least one light transmitting fibers 520. The light transmitting fibers may be arranged along the internal circumference of the hollow core fiber 510. The light transmitting fibers 520 may be of sufficient size to maintain a hollow space 530 inside of the hollow core fiber 510. The hollow core fiber 510 may integrate with a guidewire 540. The guidewire 540 may be positioned at the hollow space 530 of the hollow core fiber 510. This may allow for a user to control the direction and/or deflection of the hollow core fiber 510. Furthermore, this may allow a user to control the placement of the hollow core fiber 510, or ultrasonic energy, in relation to the treatment site. This may be advantageous for ensuring ultrasound energy is delivered to the desired treatment site. This embodiment of a fiber optic cable 410 can be used in any of the embodiments described herein.

With reference to FIG. 6, an example embodiment of an ultrasound energy emitting device 600 with a guidewire is illustrated. The device 600 includes a fiber optic cable 610, an ultrasound element 620, and a guidewire 630. The fiber optic cable 610 may be a hollow core fiber. The hollow core fiber 610 may allow for a guidewire 630 to extend through the hollow core fiber 610 and/or through the ultrasound element 620. The guidewire 630 may include nitinol. The guidewire 630 may be a 0.035″ or 0.055″ nitinol guidewire. In some embodiments, the guidewire 630 may be a 0.018″-0.1″ nitinol guidewire. The guidewire 630 may allow a user to direct and/or control movement of the hollow core fiber 610 and ultrasound element 620. This may be advantageous for ensuring the generated ultrasound energy is delivered to the intended treatment site or target.

The ultrasound element 620 may be a lens 621. As shown in FIG. 6, the lens 621 may be an optoacoustic lens. The optoacoustic lens may generate ultrasound energy when photonic energy is received by the lens. The lens 621 may be concave, convex, flat, diffuse, etc. The lens 621 may include at least one divot 640. The divot 640 may be annularly positioned on the lens 621. This may allow for ultrasound energy to be directed radially outward at the treatment site. The lens 621 may be of various shapes and sizes. As shown in FIG. 6, the lens 621 may have a shower glass diffusing face 650. The shower glass diffusing face 650 may have a jagged edge to diffuse the ultrasonic energy generated.

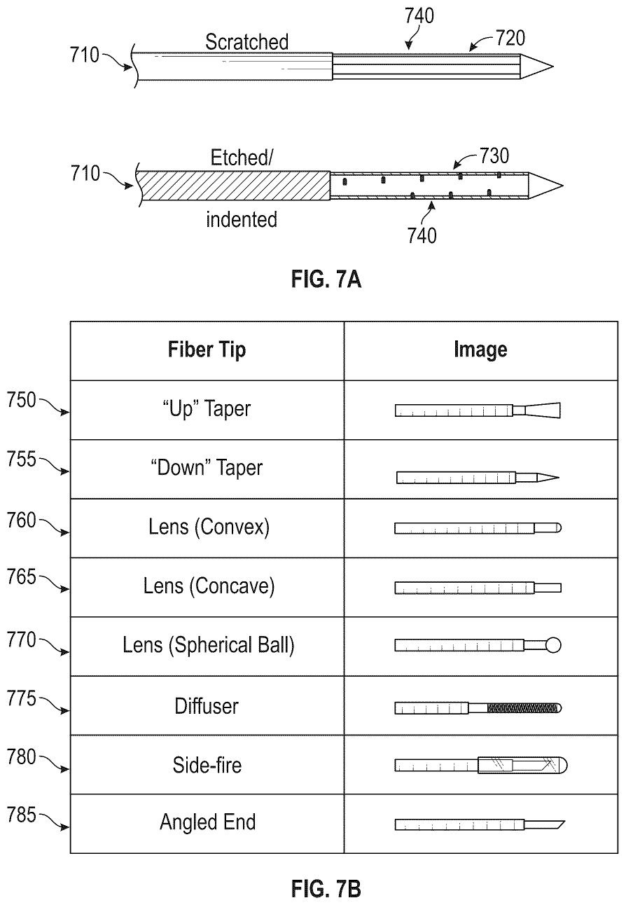

FIG. 7A illustrates an example embodiment of a fiber optic cable 710. The fiber optic cable 710 can comprise a core material of higher refractive index as compared to one or more outer layers (referred to herein as “cladding”) materials of lower refractive index which cover the core material. The cladding can cause light to be confined to the core of the fiber by total internal reflection at the boundary between the core and cladding. As shown in FIG. 7A, in certain embodiments, the fiber optic cable 710 can include cladding that is scratched 720, cladding that is etched 730, and/or indented or otherwise removed from the outer surface of the fiber optic fiber cable so as to form an exposed or partially exposed portion. In certain embodiments, the cladding can be omitted from being applied to a sections of the core to form the exposed or partially exposed section. The etchings, indentations, or scratches can extend any length of the cladding coupled to the fiber optic cable. In the illustrated embodiment, the exposed or partially exposed section 740 is formed on a distal portion or end of the fiber optic cable 710. However, as explained below, the exposed or partially exposed sections can also be formed on middle portions distanced from the ends of the fiber optic cable 710. The etchings, indentations, or scratches may be vertically or horizontally arranged. As explained above, the etchings, indentations, or scratches may allow photonic energy to exit the fiber optic cable 710 at the various etchings, indentations, or scratches and be received by the ultrasound element.

FIG. 7B illustrates an example embodiment of various fiber optic cables 710, referenced in FIG. 7A. As shown, the fiber optic cable 710 may include various tip types as defined by the outer surface shape of the cable. For example, the fiber optic cable 710 may have an “up” taper tip type 750. The “up” taper tip type 750 may be funnel-like in shape. The “up” taper tip type 750 may be configured to increase spot size to decrease power density at surface for coupling high power into the fiber optic cable. As another example, the fiber optic cable 710 may have a “down” taper tip type 755. The “down” taper tip type 755 may be conical in shape. The “down” taper tip type 755 may be configured to decrease spot size and increase divergence. As another example, the fiber optic cable 710 may have a convex lens tip type 760. The convex lens 760 may be configured to increase light collection and/or decrease light divergence. As another example, the fiber optic cable 710 may have a concave lens tip type 765. The concave lens 765 may be configured to increase light divergence. As another example, the fiber optic cable 710 may have a spherical ball lens tip type 770. The spherical ball lens tip type 770 may be configured to increase light collection. As another example, the fiber optic cable 710 may have a diffuser tip type 775. The diffuser type tip 775 may be configured to illuminate 360-degrees through the side of the fiber optic cable tip. As another example, the fiber optic cable 710 may have a side-fire tip type 780. The side-fire tip type 780 may be configured to redirect light sideways. As another example, the fiber optic cable 710 may have an angled end tip type 785. The angled end tip type 785 may be configured to reduce back reflection. In these examples, the tip of the fiber optic cable can be exposed, or partially exposed sections as described above with reference to FIG. 7A.

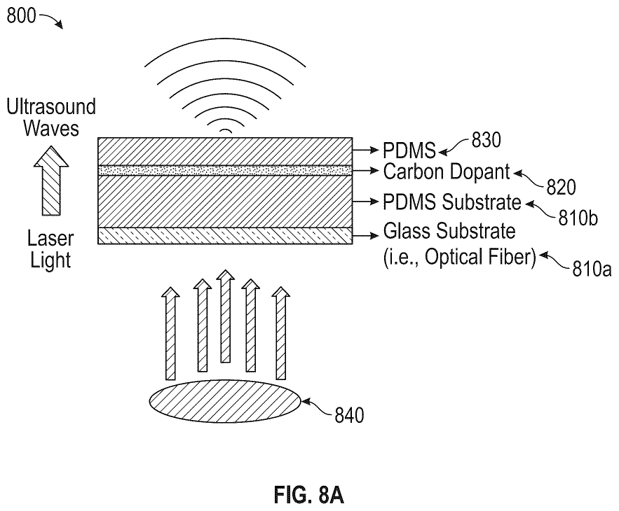

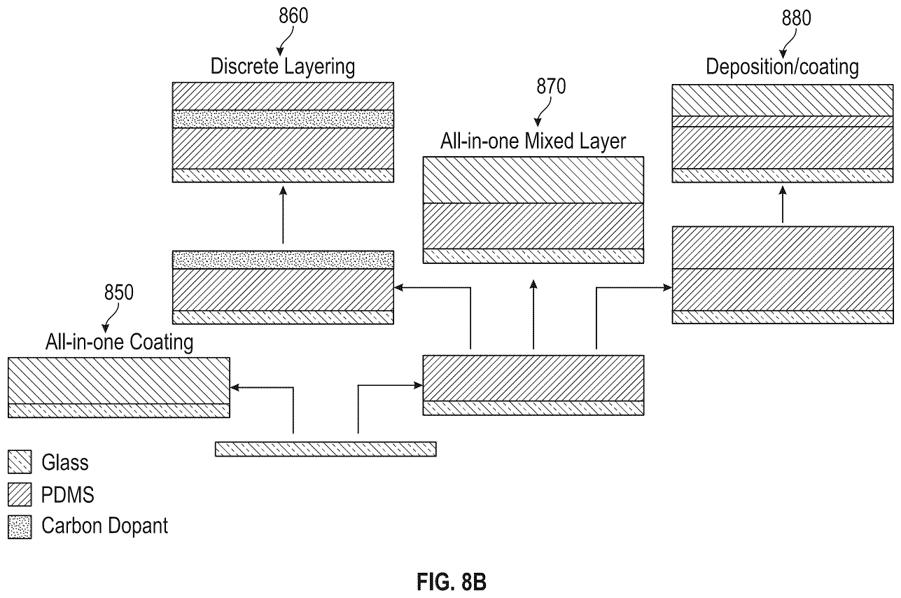

FIG. 8A illustrates an example embodiment of an ultrasound element 800 which schematically illustrates ultrasound generation according to certain embodiments. The ultrasound element 800 may include a first transmission layer 810 (which in certain embodiments, as described below, can include a portion of the optical fiber), a second absorption layer 820 and a third layer 830. The first layer 810 may be a substrate layer. The first layer 810 can be a structural layer and may provide structure to the ultrasound element 800 and in certain embodiments the first layer can also expand as heated while also facilitating the passing of light. The first layer may be made of a material that has sufficient shaping capabilities and have a high transmissivity of light (i.e., may allow for a transmission of light greater than approximately 75%). The first layer 810 may include a first substrate layer 810a and a second substrate layer 810b. The first substrate layer 810a may be a glass substrate layer and in certain embodiments may include a portion of the optical fiber. The transmission layer may also include a second substrate layer 810b that in certain embodiments can be a PDMS layer. The glass substrate layer may be at least a portion of the fiber optic cable. The first transmission layer 810 can be used to transmit light to the second layer 820 and provide structural shape to the element 800. As noted above, the second layer 820 may be an absorption layer that can absorb all or substantially all of the light transmitted from the transmission layer. As shown, the second layer 820 can be formed of a variety of materials configured to absorb light and in the illustrated embodiment may be formed of a carbon nanofiber film absorption layer. For example, the second layer can be formed of a PDMS material, EEA material, EVA material, FEP material, PB material, CA material, PVDF material, PA material, PE material, Paraffin material, or PP material. The third layer 830 may be a resonance layer. As shown, the third layer may be a PDMS resonance layer. Each of the layers can be formed into a variety of shapes (e.g. cylindrical, spherical, flat, etc.). As shown in FIG. 8A, a light source 840 may emit light (e.g., laser light, high power LED light, or any other transmission of light), which may first come in contact with the first layer 810. This may cause the photonic energy to be absorbed by the second layer 820. The absorbed photonic energy may then cause the third layer 830 to thermally expand and emit acoustic waves. In certain embodiments, it can be advantageous to control the thickness of the first layer 810 and the third layer 830. The thickness of the first layer 810 and the third layer 830 may impact or effect the resonance that the layer may operate at. The photonic energy may be used to cavitate and/or implode at least one microbubble. Therefore, it may be advantageous to manipulate the resonance of the photonic energy so that the photonic energy can be used to cavitate and/or implode at least one microbubble, but not cause damage or induce fractures to its surroundings. The ultrasound element of FIG. 8A can be combined with and applied to the exposed or partially exposed sections of the embodiments described above with reference to FIGS. 7A and 7B such that the light escaping from the exposed or partially exposed sections can be transmitted through the first layer 810.

FIG. 8B illustrates an example modified embodiments of the ultrasound element 800, as shown in FIG. 8A. As shown in FIG. 8B, the ultrasound element 800 can include a glass substrate layer, a PDMS substrate layer, an absorption layer, and a resonance layer. In certain embodiments, these layers can be formed as four distinct layers. In other embodiments, the absorption layer can be positioned in the middle but have transitions zones between the top and bottom layers. The ultrasound element 800 can have various arrangements of the first layer, second layer, third layer, and fourth layer. For example, the arrangement may be an all-in-one coating arrangement 850, a discrete layering arrangement 860, an all-in-one mixed layer arrangement 870, or a deposition/coating arrangement 880. Each layer is added to the preceding layer as an additional structure. In other words, for example, the second layer is added as an additional structure to the first layer, rather than the second layer being applied to the first layer through a dipping or coating process. Because, for example, each subsequent layer is added to the previous layer as an additional structure, each subsequent layer may also serve to cushion to the ultrasound transducer as the ultrasound transducer vibrates. Additionally, each subsequent layer may also serve as a bulking layer to the first layer. Increasing the bulkiness of the fiber optic cable may help to diffuse the ultrasonic energy. This may lead to a greater surface area, in turn a lower intensity output of energy that can be used to implode at least one microbubble to create an indirect method for treating blood clots. In other words, a negative pressure can be used to implode and/or cavitate at least one microbubble to create an indirect method for treating blood clots.

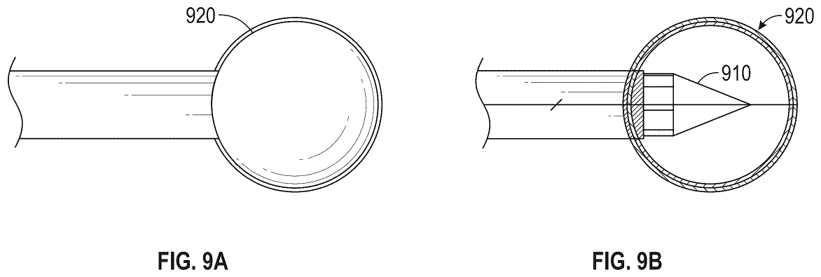

FIG. 9A and FIG. 9B illustrates an example embodiment of a fiber optic cable 910 surrounded by an ultrasound element 920. As shown, the ultrasound element 920 can be spherical in shape. In other embodiments, the ultrasound element 920 may be any shape. The ultrasound element 920 can connect to the fiber optic cable 910 near the distal end of the fiber optic cable 910. The ultrasound element 920 may have a hollow core to fit over the distal endo the fiber optic cable 910. As shown in FIGS. 9A, 9B, the ultrasound element can be positioned around the exposed or partially exposed section of the fiber optic cable 910 where the cladding has been removed. As described above with respect to FIGS. 8A and 8B, the ultrasound element 920 can include a glass substrate layer, PDMS substrate layer, an absorption layer, and a resonance layer.

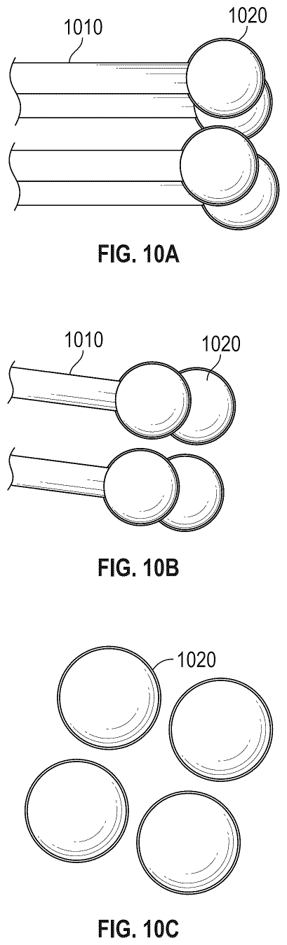

FIG. 10A, FIG. 10B, and FIG. 10C illustrate various views of a plurality of fiber optic cables 1010 each surrounded by an ultrasound element 1020. The cables and element can be arranged as described above with reference to FIGS. 9A, 9B. For example, FIG. 10A illustrates a side view of the plurality of fiber optic cables 1010 each surrounded by the ultrasound element 1020. As shown, the plurality of fiber optic cables 1010 are arranged in a non-co-planar arrangement. Each fiber optic cable 1010 may be arranged so that a distance of approximately ¼-½ an ultrasonic wavelength 2 may be between each fiber optic cable 1010. This distance may allow for adjustment of the phase relations between each ultrasound element 1020. Adjustment of the phase relations may allow for a more targeted treatment. FIG. 10B illustrates an isometric view of the plurality of fiber optic cables 1010 each surrounded by the ultrasound element 1020. As shown, the plurality of fiber optic cables 1010 are arranged in the non-co-planar arrangement. FIG. 10C illustrates a front view of the plurality of fiber optic cables 1010 each surrounded by the ultrasound element 1020. As shown, the plurality of fiber optic cables 1010 are arranged in the non-co-planar arrangement. In certain embodiments, the elements can be operated to produce a swirling or vortex flow pattern of ultrasound energy. In this manner each fiber optic cable may generate ultrasonic energy sequentially to create a swirl or vortex flow pattern of ultrasound energy.

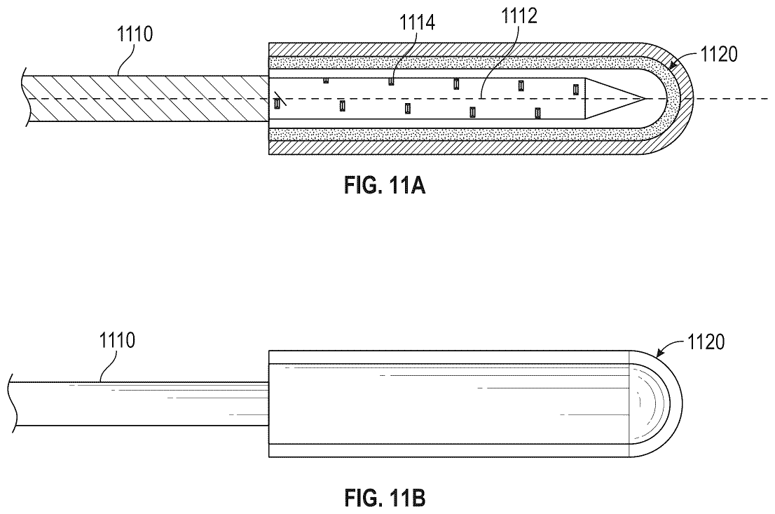

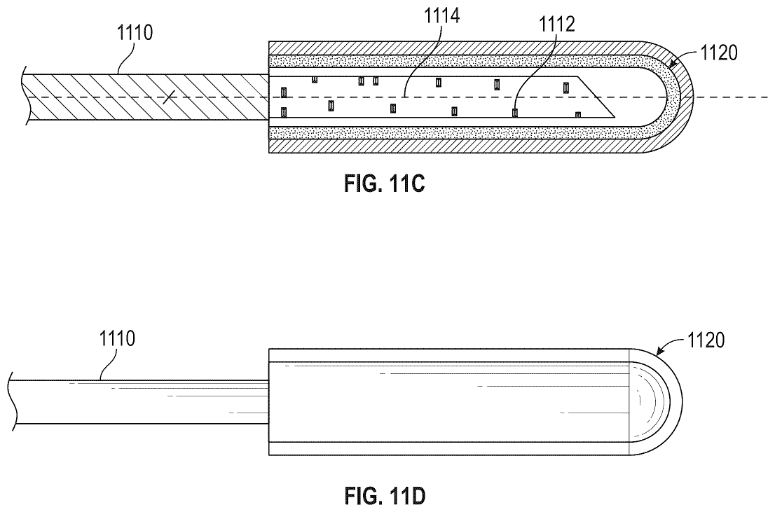

FIG. 11A, FIG. 11B, FIG. 11C, and FIG. 11D illustrate an example embodiment of a fiber optic cable 1110 surrounded by an ultrasound element 1120. As shown, the ultrasound element 1120 is an elongated sphere in shape with a generally cylindrical proximal portion and a spherical distal portion. As shown, the fiber optic cable 1110 can include cladding proximal to the ultrasound element and the ultrasound element can be positioned over exposed or partially exposed section of the fiber optic cable 1110 where the cladding has been removed. As described above with respect to FIGS. 8A and 8B, the ultrasound element 1120 can include a glass substrate layer, PDMS substrate layer, an absorption layer, and a resonance layer. In the illustrated embodiment, the cladding 1112 can include a plurality of scratches 1114 arranged along the length of the cladding 1112 beneath the element 1120. The ultrasound element 1120 may have a hollow core. As shown in FIG. 11A, the cladding may be conical in shape or form a pointed tip at the distal end of the fiber optic cable. As shown in FIG. 11C, the cladding may be angled or form an off-center tip at the distal end of the fiber optic cable.

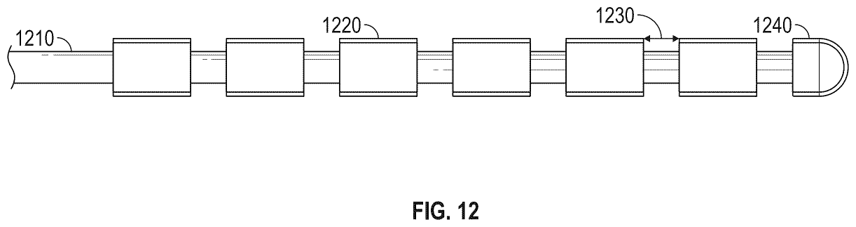

FIG. 12 illustrates an example embodiment of a fiber optic cable 1210 intermittently surrounded by a plurality of ultrasound elements 1220. The plurality of ultrasound elements 1220 may be spaced apart along the length of the fiber optic cable 1210 so that a distance 1230 is between each of the plurality of ultrasound elements 1220. The elements 1220 can be positioned over exposed or partially exposed section of the fiber optic cable 1210 where the cladding has been removed as described above. As described above with respect to FIGS. 8A and 8B, the ultrasound element 920 can include a glass substrate layer, PDMS substrate layer, an absorption layer, and a PDMS resonance layer.

The plurality of ultrasound elements 1220 may be cylindrical in shape. A final ultrasound element 1240 of the plurality of ultrasound elements 1220 may have a rounded face. The arrangement of the plurality of ultrasound elements may allow flexibility for of the fiber optic cable. Additionally, the arrangement of the plurality of ultrasound elements 1220 may be customizable dependent of the anatomy of the patient.

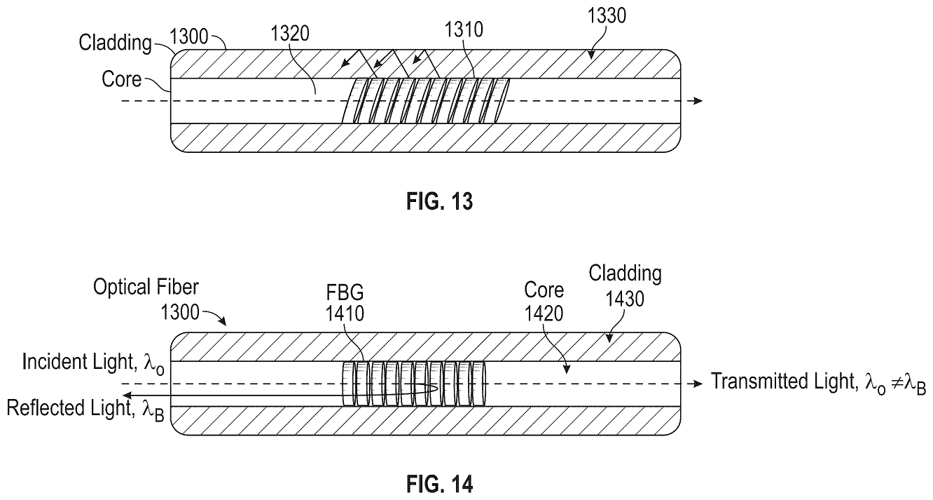

FIG. 13 illustrates an example embodiment of a fiber Bragg grating which can be used in a catheter along with the embodiments described above. The fiber Bragg grating construct may be any kind of construct, for example, chirped, uniform, tilted, superstructure, etc. As shown in FIG. 13, the fiber Bragg grating 1310 construct can be tilted. The fiber Bragg grating 1310 may be located within a core 1320 of a fiber optic cable 1300 of which is surrounded by cladding 1330. The fiber Bragg grating 1310 may be used simultaneously with or complimentary to the fiber optic cable including the laser generated ultrasound element. For example, the fiber optic cable 1300 that includes the fiber Bragg grating 1310 may extend through a catheter. The fiber optic cable including the laser generated ultrasound element may also extend through the catheter. The fiber Bragg grating 1310 may be in a separate fiber that extends through the catheter before, after, or at the same time that the fiber optic cable that includes the laser generated ultrasound element may extend through the catheter. The fiber Bragg grating could also be added to a fiber optic cable that is coupled to an ultrasound element as described above. The Bragg grating 1310 may provide a feedback control for the laser and laser generated ultrasound element that extends through the catheter. For example, the fiber Bragg grating 1310 may monitor a temperature of the blood and/or tissue surrounding the insonation zone, which as explained above, may help to avoid overheating of the tissue, blood, and/or laser. Additionally, this may allow the device to be driven closer to the target site and therefore provide a more directed treatment to the target site. This can provide may benefits to the treatment, including but not limited to, reducing the overall time of the procedure, reducing the amount of drugs delivered to the target site, reducing the risk of damage caused to surrounding tissues, increasing the efficiency of the treatment, etc. Additionally, the fiber Bragg grating 1310 may also monitor pH, flow, pressure, force, torsional measurements, a distance between the device and/or catheter and the target site, color reflectance spectroscopy, flex of the fiber optical fiber 1300, chemical fluorescence, etc. Furthermore, the fiber Bragg grating 1310 may monitor the pressure or force that the fiber optic cable 1300 is applying to the target site. This may help to ensure the fiber optic cable 1300 is not damaging surrounding tissue. The fiber Bragg grating 1310 may also provide feedback regarding the color reflectance spectroscopy of the surrounding blood or tissue. For example, the fiber Bragg grating 1310 may provide feedback regarding the blood color (SᵥO₂), the condition of the clot including but not limited to the age, degradation, thickness, strength, toughness, etc. of the target tissue. The fiber Bragg grating 1310 may also assist in guiding the device to the target site and provide feedback regarding the tortuosity of the vessel that the device may travel through. Additionally, the fiber Bragg grating 1310 may also detect gas released during inertial cavitation of the microbubbles, chemicals related to lysis, etc.

FIG. 14 illustrates an example embodiment of a fiber Bragg grating 1410 as described with reference to FIG. 13. The fiber Bragg grating 1410 construct may be any kind of construct, for example, chirped, uniform, tilted, superstructure, etc. As shown in FIG. 14, the fiber Bragg grating construct is uniform. The fiber Bragg grating 1410 may be located within the core 1420 of the fiber optic cable 1400 of which is surrounded by cladding 1430. The fiber Bragg grating 1410 may be used simultaneously with or complimentary to the fiber optic cable including the laser generated ultrasound element. For example, the fiber optic cable 1300 that includes the fiber Bragg grating 1410 may extend through a catheter. The fiber optic cable including the laser generated ultrasound element may also extend through the catheter. The fiber Bragg grating 1410 may extend through the catheter before, after, or at the same time that the fiber optic cable that includes the laser generated ultrasound element may extend through the catheter. The location of the fiber Bragg grating 1410 may allow for more accurate and precise monitoring of the overall treatment delivered to the patient.

### Terminology

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. The terms “comprising,” “including,” “having,” and the like are synonymous and are used inclusively, in an open-ended fashion, and do not exclude additional elements, features, acts, operations, and so forth. That is, the above terms are to be interpreted synonymously with the phrases “having at least” or “including at least.” For example, when used in the context of a process, the term “comprising” means that the process includes at least the recited steps but may include additional steps. When used in the context of a device, the term “comprising” means that the device includes at least the recited features or components, but may also include additional features or components. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The term “and/or” as used herein has its broadest least limiting meaning which is the disclosure includes A alone, B alone, both A and B together, or A or B alternatively, but does not require both A and B or require one of A or one of B. As used herein, the phrase “at least one of” A, B, “and” C should be construed to mean a logical A or B or C, using a non-exclusive logical or.

Any methods disclosed herein need not be performed in the order recited. The methods disclosed herein include certain actions taken by a practitioner; however, they can also include any third-party instruction of those actions, either expressly or by implication.

Conditional language used herein, such as, among others, “can,” “could,” “might,” “may,” “e.g.,” and the like, unless specifically stated otherwise, or otherwise understood within the context as used, is generally intended to convey that certain, certain features, elements and/or steps are optional. Thus, such conditional language is not generally intended to imply that features, elements and/or steps are in any way required or that one or more implementations necessarily include logic for deciding, with or without other input or prompting, whether these features, elements and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain implementations require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

Any methods disclosed herein need not be performed in the order recited. The methods disclosed herein include certain actions taken by a practitioner; however, they can also include any third-party instruction of those actions, either expressly or by implication.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain implementations disclosed herein is indicated by the appended claims rather than by the foregoing description. All changes which come within the meaning and range of equivalency of the claims are to be embraced within their scope.

What is claimed is:

1. A device configured to emit ultrasound energy comprising:
a catheter body;
a fiber extending through the catheter body from a proximal portion of the catheter body to a distal portion of the catheter body; and
an ultrasound element positioned coupled to the fiber configured to receive photonic energy from the fiber, the ultrasound element having a side surface that faces a generally lateral direction with respect to a longitudinal axis of the catheter body;
the side surface including at least one protrusion or recess that directs ultrasound energy laterally from the longitudinal axis of the catheter body.

2. The device of claim 1, wherein the fiber connects to a light source to transmit photonic energy.

3. The device according to any one of claim 1-2, wherein the ultrasound element is a lens.

4. The device of claim 3, wherein the lens is a PDMS lens.

5. The device according to any one of claim 3-4, wherein the lens is concave.

6. The device according to any one of claim 3-4, wherein the lens is convex.

7. The device according to any one of claim 3-4, wherein the lens is flat.

8. The device according to any one of claim 3-4, wherein the lens is diffuse.

9. The device according to any one of claim 3-4, wherein the lens forms an annular ring.

10. The device according to any one of claim 1-9 further comprising a hollow core fiber located at a center of an arrangement, the arrangement comprising the fiber.

11. A method to deliver ultrasonic energy to a treatment site, the method comprising:
transmitting photonic energy from a light source through a fiber optic cable;
generating ultrasound energy from the transmitted photonic energy at an ultrasound element connected to the fiber optic cable;
penetrating a blood clot with forward firing ultrasonic energy from the ultrasound element; and
radially firing the ultrasound energy from a side surface of the ultrasound element.

12. A device configured to emit ultrasound energy, the device comprising:
a catheter body;
a fiber optic cable extending through the catheter body from a proximal portion of the catheter body to a distal portion of the catheter body; and
an ultrasound element coupled to the fiber optic cable configured to receive photonic energy from the fiber optic cable, wherein the ultrasound element comprises a substrate layer, an absorption layer and a resonance layer, the substrate layer comprising a structural material, the absorption layer comprising a light-absorbing material and the resonance layer comprising a thermal expansion material and wherein the structural layer is positioned closer to the fiber optic cable and the absorption layer is positioned between the structural and resonance layer.

13. The device of claim 12, wherein at least a portion of the fiber optic cable is in a vacuum.

14. The device according to any one of claim 12-13, wherein the substrate layer receives photonic energy from the fiber optic cable and causes the resonance layer to emit a plurality of ultrasonic waves.

15. The device according to any one of claim 12-14, wherein the substrate layer is at least one of a glass material, an optical fiber core, or a PDMS material.

16. The device according to any one of claim 12-15, wherein the resonance layer is a PDMS material.

17. The device according to any one of claim 12-16 further comprising at least one clad surrounding the fiber optic cable.

18. The device of claim 17, wherein the clad has at least one divot configured to allow light to escape the fiber optic cable.

19. The device of claim 18, wherein the light escapes the fiber optic cable in a forward and lateral direction.

20. The device according to any one of claim 12-19 further comprising a plurality of ultrasound elements positioned along the fiber optic cable.
